(19) United States
(12) Patent Application Publication
(10) Pub. No.: US 2019/0209386 A1
(43) Pub. Date: Jul. 11, 2019

(54) Protective Cover for a Walking Brace, Cast and/or Orthotic Device

(71) Applicants: Kristopher Fequet, Gatineau (CA); Hayley Sprigings, Ottawa (CA); Shaliza Merchant, Amsterdam (CA)

(72) Inventors: Kristopher Fequet, Gatineau (CA); Hayley Sprigings, Ottawa (CA); Shaliza Merchant, Amsterdam (CA)

(21) Appl. No.: 15/868,919

(22) Filed: Jan. 11, 2018

(51) Int. Cl. A61F 13/04; A61F 15/00

(52) U.S. Cl. CPC A61F 13/043; A61F 5/01; A61F 15/004

(57) ABSTRACT

The present invention is a weather-resistant cover for a walking brace, cast, and/or orthotic device used on a human leg and foot. This innovative cover includes a sturdy, slip-resistant outer sole for safe mobility. The majority of the protective cover is comprised of a flexible, resilient, and weather-resistant material. In contrast, patches of flexible, thin fabric are present over the regulatory buttons of the walking brace, cast, and/or orthotic device. These patches allow for the fitting of the walking brace, cast, or orthotic device to be adjusted while the cover is in use. A drawstring is present at the top of the cover to ensure the cover is completely weather-resistant. This cover also includes a longitudinal fastener at the rear of the cover to allow the wearer to easily place their leg/foot in the device. This fastener, when closed, secures the wearer's appendage in the cover.

### FIELD

The present invention relates to weather protectors for a human leg and foot in a walking brace, cast, and/or orthotic device.

### BACKGROUND OF THE INVENTION

Protective covers for walking braces, casts, and/or orthotic devices have been used for many years. Several covers are known for protecting a human leg and foot, separately or as a whole, that have been placed in a walking brace, cast, and/or orthotic device. The current available protective covers for these devices have various flaws.

A casted leg or arm were previously protected by a cast protector, for example U.S. Pat. No. 7,955,284 B2, but were limited to providing protection while taking showers and baths. This protector was entirely waterproof, but did not allow the wearer to safely walk using the cover, as it did not have a non-slip sole.

U.S. Pat. No. 7,475,501 B1 is an example of a cover for a walking brace, cast, and/or orthotic device which was designed to be weather-resistant. In contrast to the previously mentioned protector, this protective cover contained an outer sole for walking in various weather conditions. The protector was composed of a waterproof material, but had one major flaw. This cover was only large enough to shield up to the wearer's ankle region, therefore the entire walking brace, cast, and/or orthotic device would not be completely protected.

These walking braces, casts, and/or orthotic devices can also be damaged by contact with physical objects. Some of these devices do not provide protection to the entire foot, specifically the most anterior portion of the forefoot. When walking, especially outdoors, it is important to protect both the entire patient's foot and the anterior portion of the device. U.S. Pat. No. 3,487,830 details a device which solely protects the anterior portion of the patient's foot in a cast. This protective cover does not provide protection to the any other portion of the patient's foot/leg in a walking brace, cast, and/or orthotic device.

Another major issue with the protective covers for walking braces, casts, and/or orthotic devices, is that many do not allow for the device to be adjusted while the protective cover is on. U.S. Pat. No. 4,217,706 is an example of a device which when protecting the walking brace, cast, and/or orthotic device, said device can no longer be adjusted in any way.

Currently there is a need for a protective cover for walking braces, casts, and/or orthotic devices which are waterproof, allow for the device to be adjusted, and contains a rigid sole.

### BRIEF SUMMARY OF THE INVENTION

The present device was invented to surround all portions of walking braces, casts, and/or orthotic devices, to allow the wearer to freely move in any environment. This protective cover is composed of a weather-resistant, resilient, natural and/or synthetic fiber such as neoprene. This material allows for the cover to tightly fit onto the walking brace, cast, and/or orthotic device, without causing any damage to said device, or interfering with the purpose of said device.

The portion of the protective cover which shields the most anterior portion of the forefoot, contains a rigid material for additional protection.

The protective cover also contains a thin, flexible material to encompass the buttons which adjust the volume in the interior of walking braces, casts, and/or orthotic devices.

The protective cover has a vertical fastening device along the entire posterior region. Furthermore, a drawstring is present along the uppermost rim of the protective cover to ensure the waterproof cover encompasses the entirety of the medical device.

An inner and outer sole are also present on the protective cover. The inner sole is comprised of multiple layers of foam, with the uppermost layer being soft, and the lowermost layer being the most firm. The outer sole being rigid with a slip-resistant tread. The sole also has a portion between the ball of the foot and the heel which does not come into contact with the surface in which the patient is walking. The following detailed description, along with its corresponding drawings, will detail supplementary and/or additional information regarding the present invention.

### BRIEF DESCRIPTION OF THE DRAWINGS

FIG. 1 is a side elevational view of the present invention

FIG. 2 is a cross-sectional view in which a patient's leg/foot in an orthotic brace has been included for illustration purposes.

FIG. 3 is a rear elevational view of the present invention

FIG. 4 is a bottom view of the sole of the present invention

### DETAILED DESCRIPTION OF THE INVENTION

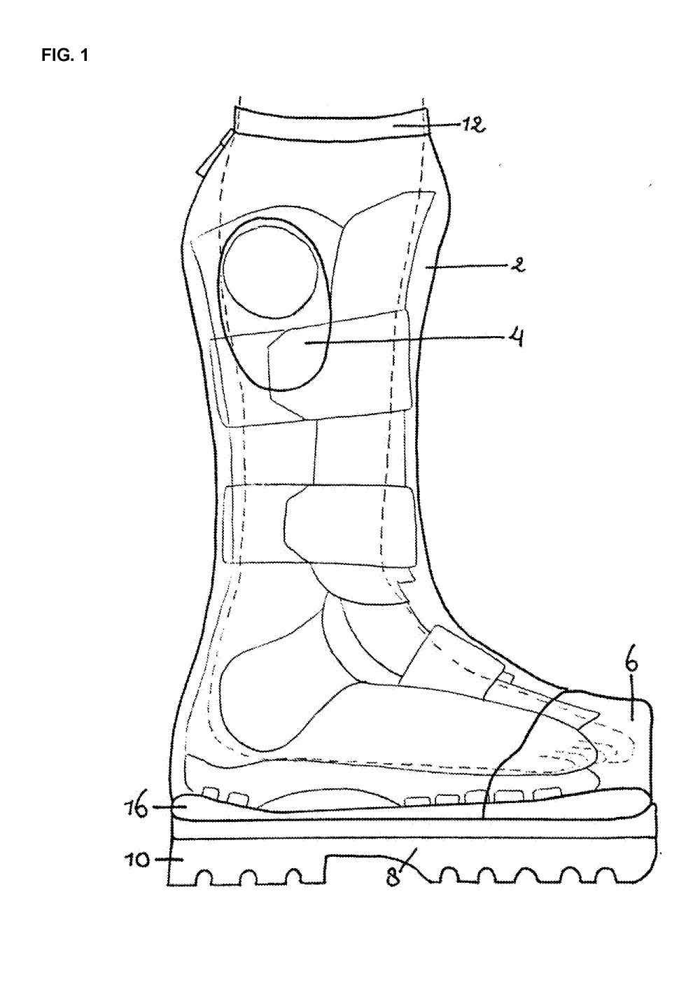

The present invention is a protective, weather-resistant cover for a cast, brace, or orthotic device. This cover protects the cast, brace, or orthotic device under various weather-conditions, while simultaneously allowing the wearer to walk without risking further injury due to slipping. FIG. 1 illustrates one preferred embodiment of the present invention. The cover, generally, is meant for a human leg and foot in a brace, cast, or an orthotic device, to be easily yet repeatedly, inserted into the cover, with little effort. One of the main components of the cover is its weather-resistant, outer body portion 2. An example of a material suitable for its manufacture is neoprene.

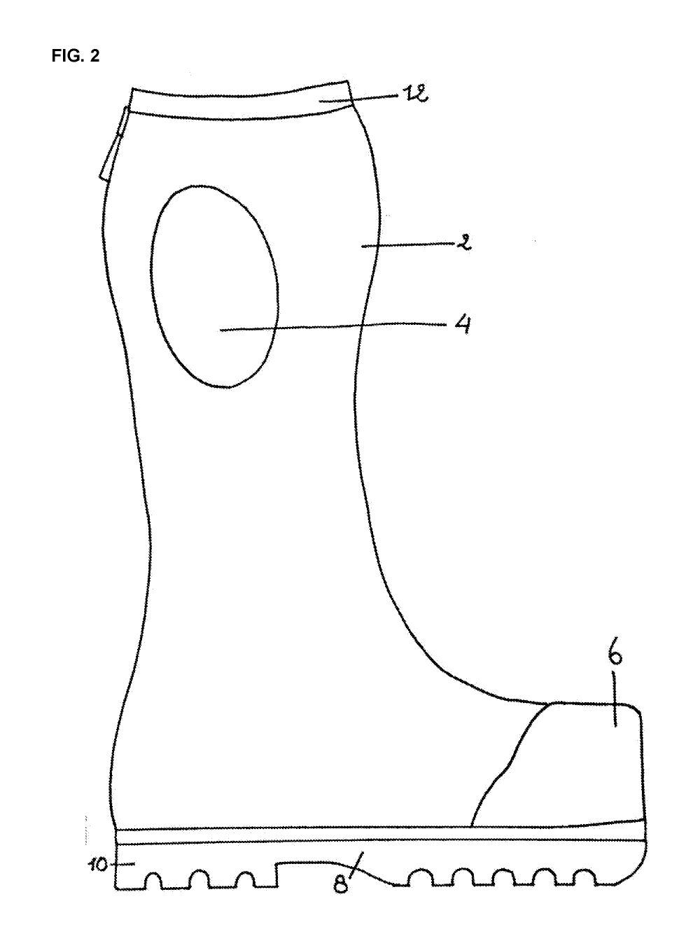

As best seen in FIG. 2, the base of body portion 2 includes inner sole 16. One suitable material for making the inner sole is foam cushion based, containing a soft nylon top layer with multiple layers of foam of different firmness. With such an inner sole on which to walk, this invention is able to accommodate various leg/foot treating devices, i.e., those having a flat, as well as those having a rounded, outer sole. The inner sole 16 prevents shearing of the foot/brace complex during walking.

Extending upwards from the front end of inner sole 16 is an enclosed toe section 6. The toe cover is squared with rounded edges. The toe cover is attached to the front portion of the outer embodiment 2 by any suitable means for example stitching. It comprises of a hard, lightweight plastic, which covers all the toes in their entirety. A squared off, front toe cover allows for a universal application, in that the model of the cover can be used over a brace and on either the wearer's right or left foot.

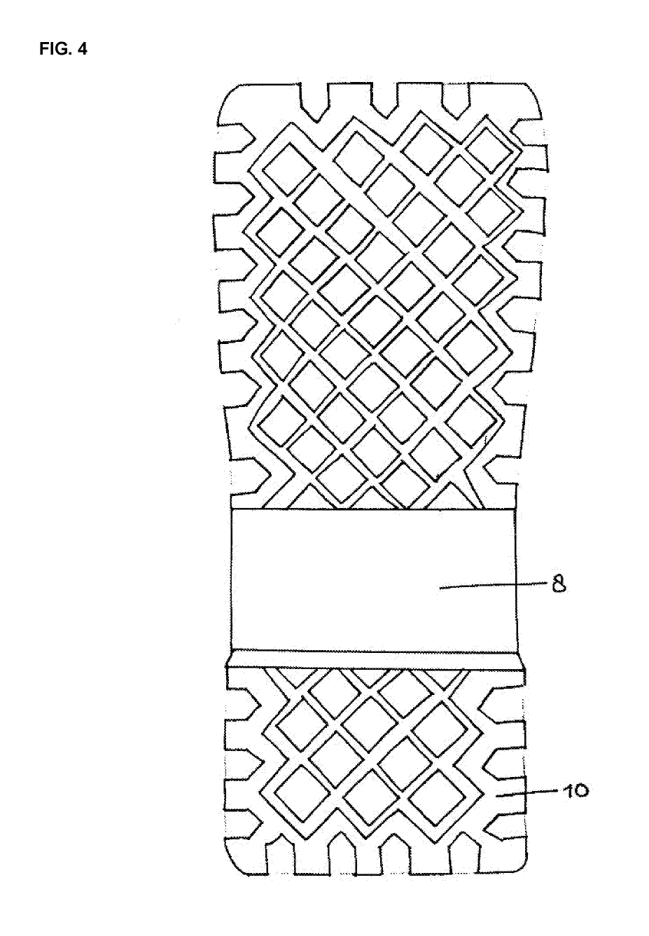

As seen in FIG. 4, a non-skid, rubber outer sole 4 is affixed to the bottom of the cover. This outer sole 8 is composed of a durable, semi-flexible material. The exposed bottom face of the outer sole 8 is adapted to frictionally engage the ground. This sole contains a roughened surface which is comprised of a plurality of projections extending outwardly from the bottom face. The rear portion of the outer sole 8 has an affixed heel portion 10 with the same roughened surface, which comprises of a plurality of projections extending outwardly from the bottom face.

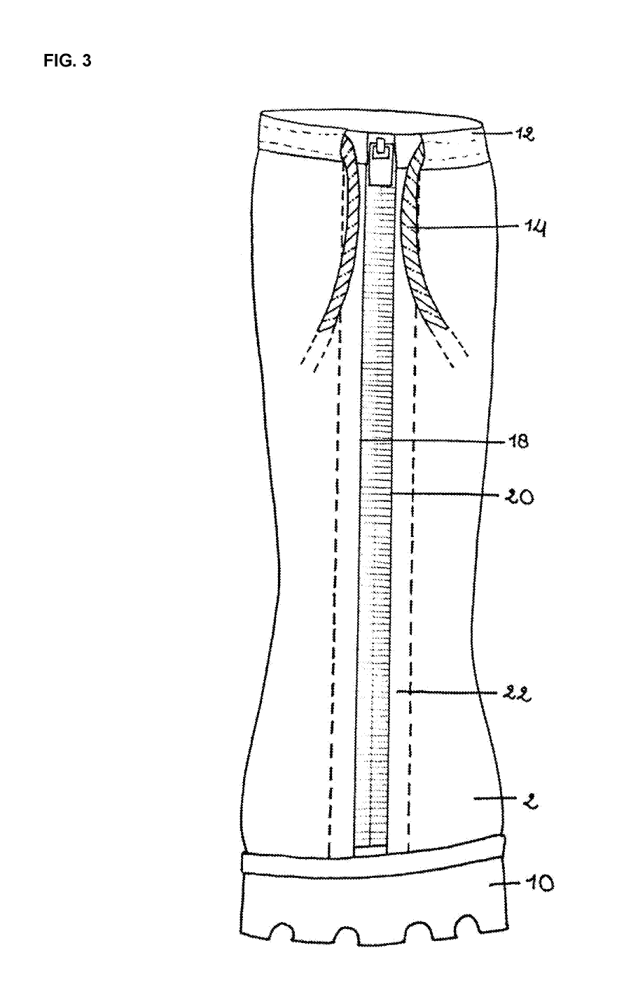

The body portion 2 is tubular in shape and extends upwardly along the lower leg. This body portion is roughly symmetrical, with a longitudinal fastener 22 placed at the midpoint of the rear of the tubular leg portion, as seen on FIG. 3. This longitudinal fastener 22 contains two strips 18 and 20 mounted to opposing sides of a slit at the posterior of the body portion 2. The fastening strips 18 and 20 are adapted to be interlocked, or parted into the open position. The fastener can open the slit so that the walking brace, cast, and/or orthotic device can be quickly and easily inserted onto the inner sole portion. When the fastener is closed, and the cover is around the walking brace, cast, and/or orthotic device, the wearer can freely walk on all surfaces.

The button cover patches 4 depicted in FIG. 1 are located on the upwardly extended integral tubular leg portion of the cover. The patches are composed of a more flexible, yet resilient material, which has the capability to provide protection against multiple weather conditions. This button covering material is on the upper lateral portion of the cover. This covering is circular and is sewn in continuity with the neoprene material. The circumference of this patch is slightly larger than the circumference of the button, as this allows the optimal trade-off by gaining access to the button over the cover, yet not compromising the support of the cover. This option allows the injured to have access to this button on the cast without having to remove the cover.

Attached to the top of body portion 2 is an additional extremely flexible, weather-resistant fabric, that is anchored to this upper body portion. This material will create a tube-like structure 12 around the top of the body portion 2. This tube 12 contains a string 14, which can be tied in a snug position around the wearer's leg to a desired tightness. This adjustment prevents any material or debris from coming into contact with the walking brace, cast, and/or orthotic device, while wearing said cover.

The following description for the preferred embodiment of a device to cover all portions of walking braces, casts, and/or orthotic devices, which allows the patient to freely move in any environment, is merely exemplary in nature. This description is in no way intended to limit the scope of the invention, but rather the scope is depicted in the claims.

REFERENCES CITED
US PATENT DOCUMENTS

| | | | |
|---|---|---|---|
| 7,955,284 B2 | May 2011 | Brown et al. | 602/3 |
| 7,475,501 B1 | July 2006 | Detoro et al. | 36/110 |
| 3,487,830 | January 1970 | Frank | 602/11 |
| 4,217,706 | January 1979 | Vartanian | 36/110 |

1. A protective device for the leg/foot that has been placed in a cast, brace, or orthotic device comprising of:
a. A fitted, weather-resistant material encompassing the entire cast, brace, or orthotic device
b. A second rigid material covering the most anterior portion of the forefoot
c. A thin, flexible material solely covering the pushbutton(s) on the brace
d. A fastening device connecting the rear of the hind foot to the rear of the calf just above the cast, brace, or orthotic device
e. A multi-layered inner sole
f. A semi-flexible, non-slip outer sole

2. Wherein the protective device described in claim 1, is comprised of a flexible, resilient, weather-resistant material that covers the entirety of the cast, as the material wraps around all contours of the cast and foot.

3. Wherein the material described in claim 2, is made of natural and/or synthetic fiber such as neoprene.

4. Wherein the forefoot plastic layer in claim 1 is composed of lightweight plastic such as carbon fiber.

5. Wherein the protective device's rigid anterior forefoot layer in claim 4, will be attached to the weather-resistant, flexible material by strong composite adhesive, which will be applied along all the edges of the hard plastic, providing a strong and sealed attachment over the toe portion covering device.

6. Wherein the protective device's rigid anterior forefoot layer in claim 4, comprises of a hard lightweight material which covers the entire anterior forefoot, except for the bottom, which is protected by the soles.

7. Wherein the outer sole mentioned in claim 1 is comprised of a non-slip material which includes a lugged surface.

8. Wherein the non-slip outer sole in claim 7, contains deep indentations and protruding elements to provide increased traction to multiple surfaces under various weather conditions.

9. Wherein the inner sole mentioned in claim 1, covers the entire interior of the bottom of said cover.

10. Wherein the foam cushion insole mentioned in claim 9, contains a soft nylon top layer with multiple layers of foam of different firmness, with increasing firmness from the insole's top foam layer to the bottom layer.

11. Wherein the material covering the braces button(s) described in claim 1, will be attached to the primary material described in claims 2 and 3 in a continuous manner.

12. Wherein the material described in claim 11, will be a circular patch covering the corresponding button, such that the surface area of the patch is larger than the surface area of the respective button.

13. Wherein the material described in claim 11, will be composed of a thin natural or synthetic polymer such as rubber.

14. Wherein the fastening device described in claim 1, will seal the cover from its rear as the fastener runs in the vertical direction from the base of the heel, along the calf, to the top of the protective cover.

15. Wherein the protective brace covering device described in claim 1, contains an additional fabric attached to the material depicted in claims 2 and 3 in the configuration of a tube surrounding the circumference of the top of the cover.

16. Wherein the additional tubular shaped fabric described in claim 15, extends from the top of the brace cover at a minimal height.

17. Wherein the tubular shaped fabric described in claim 15, has a singular, flexible string running through the tube's interior.

18. Wherein the string described in claim 17 runs through the tube and exits the tube through two perforations near the fastening strips.

19. Wherein the tubular shaped fabric described in claim 16, is constructed using a flexible weather-resistant material such as polyvinyl chloride.
